(19) United States
(12) Patent Application Publication
(10) Pub. No.: US 2024/0052289 A1
(43) Pub. Date: Feb. 15, 2024

(54) SEPARATING SYSTEM

(71) Applicant: CELL THERAPY CATAPULT LIMITED, London, Greater London (GB)

(72) Inventors: Nick GADDUM, London, Greater London (GB); Jahid HASAN, London, Greater London (GB); Mohsen SHAERI, London, Greater (GB)

(21) Appl. No.: 18/267,868

(22) PCT Filed: Dec. 13, 2021
(86) PCT No.: PCT/GB2021/053268

§ 371 Date: Jun. 16, 2023

(30) Foreign Application Priority Data
Dec. 18, 2020 (GB) 2020182.8

(51) Int. Cl. C12M 1/00; C12M 1/26; B04C 5/13

(52) U.S. Cl. CPC C12M 47/02; C12M 33/10; B04C 5/13; B04C 2005/133

(57) ABSTRACT

A separating system, for example for separating material from a suspension such as a biological suspension, is disclosed herein. The system comprises a separation vessel arranged to enable the formation of a cyclone therewithin. For example, the separation vessel may be at least partially conical in shape for enabling the formation of a cyclone therewithin. The separation vessel comprises a fluid inlet, an underflow outlet, a first overflow outlet for removing fluid from a first region inside the separation vessel, and a second overflow outlet for removing fluid from a second region inside the separation vessel.

### FIELD OF THE INVENTION

The present disclosure relates to a biological suspension separating system, method and controller, such as a system, method and controller for separating cells from a biological suspension.

### BACKGROUND

The expansion of cells may be desirable for many applications in the general sphere of biotechnology. These include cell culture in the context of fermentation, as well as the use of cells in the manufacture of other biological products derived from the cells, such as small molecules, biologically active factors, proteins (secreted or otherwise), monoclonal antibodies, or other cell derived products. More recently, vaccines or gene therapies requiring the use of cells for the manufacture of viral particles or viral vector particles have emerged, necessitating the expansion of a population of cells for such manufacture. Similarly, the expansion of cells is required for the manufacture of cell therapies, for example in both allogeneic and autologous cell therapies (including stem cell-based therapies), to allow the generation of product to meet patient demand.

Depending on the application the cells may be of any type, for example the cells may be eukaryotic or prokaryotic. The cells may have been grown in suspension (e.g. freely or cultured on microcarriers), or in a two-dimensional system and then released from their substrate for further processing prior to being separated by the technology of the present invention.

Media replacement is a requirement for many cell culture systems, to allow optimal product development and yield, and the use of efficient cell culture perfusion systems, which allow the maintenance of a specific environment in a culture vessel or bioreactor, is important for the production of cell therapies. Additionally, there exists a requirement to concentrate cells after expansion into an appropriate volume for downstream applications to enable product formulation. Particularly, cell therapies often require the culture and expansion of cells from a single individual, for ultimate administration to multiple patients, necessitating intensified cell expansion (perfusion) and an ultimate formulation (volume reduction).

Furthermore, there is a requirement in some applications for the harvest of products derived (e.g. secreted or otherwise) from the cells, (such as small molecules, biologically active factors, proteins, antibodies, exosomes or viral vector particles) from the cell culture medium. In such circumstances the cell culture medium comprises the product of interest to be harvested, and the cells may be reused for continuous expansion and/or manufacture.

Hydrocyclones are devices which are commonly used to separate or sort particles from a fluid suspension, typically involving the formation of a cyclone to achieve separation of larger and/or denser material from the fluid in which it is suspended. The devices are very simple with no moving parts, are robust and sterilisable in situ. However, their use in real life can be problematic due to the different conditions and environments in which they are used affecting the formation of a cyclone inside the device, and therefore the efficacy of such devices, requiring careful balancing and fine tuning every time a change is made to the system. The use of hydrocyclones is particularly problematic when scaling up where the use of large volumes of suspension can result in a low yield or recovery of product.

### SUMMARY OF THE INVENTION

Aspects of the invention are as set out in the independent claims and optional features are set out in the dependent claims. Aspects of the invention may be provided in conjunction with each other and features of one aspect may be applied to other aspects.

A typical hydrocyclone device in use comprises a pump pumping in a fluid through an inlet into a separation vessel. The separation vessel may be elongate and symmetrical (for example, the separation vessel may have a conical or tubular shape) and therefore have a longitudinal axis extending in the elongate direction. The fluid is fed into the separation vessel transverse to (for example, perpendicular to) and eccentric to a longitudinal axis of the separation vessel such that it creates a cyclone effect in the vessel centred about (and therefore coaxial to) the longitudinal axis. Typically, denser and/or more massive particles in the fluid travel around the sides of the separation vessel and out through an outlet at one end of the separation vessel (the underflow outlet), and fluid comprising fewer particles, or less dense particles with a smaller mass, are forced through an outlet at another end of the separation vessel (the overflow outlet).

However, in the present case the inventors have surprisingly discovered that when a separating system is used for the separation of biological suspensions, two vortices are formed inside the separation vessel, one nested within the other and rotating in an opposite direction to the other. What this means is that when the cyclone is formed (comprising both vortices), material that is suspended in the suspension is forced both upwards along the longitudinal axis of the cyclone towards one of the overflow outlets (by one of the vortices) and downwards along the longitudinal axis of the cyclone towards the underflow outlet (by the other of the vortices). Therefore, to increase the concentration of material such as cells in the suspension, a second overflow outlet is used to extract the suspending solution (such as cell media) from the “eye” of the cyclone, as can be seen for example in FIG. 3B.

Accordingly, in a first aspect there is provided biological suspension separating system, for separating material (e.g. cells and/or beads) from a suspension, particularly from a biological suspension, such as a cell suspension. The system may be for concentrating cells in cell media. The system comprises a separation vessel arranged to enable the formation of a cyclone therewithin. For example, the separation vessel may be at least partially conical in shape for enabling the formation of a cyclone therewithin. However, it will be understood that the formation of a conical cyclone in the separation vessel may not be essential for enabling separation to occur. The separation vessel comprises a fluid inlet for delivering fluid to the vessel, an underflow outlet for removing fluid from the vessel, a first overflow outlet for removing fluid from a first region inside the separation vessel, and a second overflow outlet for removing fluid from a second region inside the separation vessel.

The system may further comprise a feed vessel, wherein the fluid inlet, the underflow outlet and the first overflow outlet are coupled to the feed vessel, a fluid flow control means coupled to the fluid inlet for delivering fluid to the separation vessel from the feed vessel, and a fluid flow control means coupled to the second overflow outlet for removing fluid from the separation vessel.

The location at which the second overflow outlet removes fluid from the vessel may be adjustable.

The separation vessel has a longitudinal axis, and wherein the first overflow outlet may be configured to draw fluid from the separation vessel at a proximal portion of the longitudinal axis and the underflow outlet is configured to draw fluid from the separation vessel at a distal portion of the longitudinal axis, and wherein the second overflow outlet may be configured to draw fluid from the separation vessel at a location between the first overflow outlet and the underflow outlet along the longitudinal axis.

The location at which the second overflow outlet removes fluid from the separation vessel may be adjustable along the longitudinal axis of the separation vessel. The fluid inlet may be configured to deliver fluid to the separation vessel at a proximal portion of the separation vessel. The first overflow outlet may be configured to draw fluid from the separation vessel at a distance along the longitudinal axis proximate to the point at which the fluid inlet delivers fluid to the separation vessel.

In some examples the first overflow outlet comprises a tube having a lumen therethrough for drawing fluid from the separation vessel, and wherein the second overflow outlet comprises a tube having a lumen therethrough for drawing fluid f rom the separation vessel, and wherein at least a portion of the tube of the second overflow outlet is configured to sit within at least a portion of the lumen of the first overflow outlet tube, and wherein at least a portion of the second overflow outlet tube is coaxial with at least a portion of the first overflow outlet tube. The tube of the second overflow outlet may be slidable within the lumen of the tube of the first overflow outlet. The position of the tube of the second overflow outlet may be adjustable relative to the position of the tube of the first overflow outlet. At least a portion of the second overflow outlet tube may be coaxial with at least a portion of the first overflow outlet tube about the longitudinal axis of the separation vessel. The tube of the first overflow outlet may extend into the separation vessel proud of an interior surface of the separation vessel, and wherein the tube of the second overflow outlet may extends into the separation vessel proud of the interior surface of the separation vessel to a greater extent than the tube of the first overflow outlet.

It will be understood that when reference is made to the terms underflow outlet and overflow outlet, these are described with reference to the separation vessel and in particular, in use, to the longitudinal axis of a cyclone formed in the separation vessel and therefore with reference to where fluid/material is extracted from the separation vessel along the longitudinal axis of the cyclone. As such, the underflow outlet may be described as an outlet that is configured to draw fluid from one point along the longitudinal axis (such as proximal to one end of the longitudinal axis), and the overflow outlet may be described as an outlet that is configured to draw fluid from a second point along the longitudinal axis (such as proximal to the other end of the longitudinal axis, and distal to the underflow outlet). It will be understood that typically the fluid inlet is located somewhere along the longitudinal axis of the cyclone, typically in use proximal to the overflow outlet at a distal end of the longitudinal axis of the cyclone. It will also be understood that the longitudinal axis of the cyclone typically corresponds to (for example, is parallel to and coaxial with) the longitudinal axis of the separation vessel, which is also an axis of symmetry of the separation vessel, particularly in examples where the separation vessel is conical or cylindrical. It will also be understood that one of the outlets may have a larger diameter (for example to accommodate a greater flow rate) than the other. For example, the first overflow outlet may be have a larger diameter than the second overflow outlet.

In some examples it will be understood that the separation vessel may comprise a plurality of fluid inlets and/or a plurality of underflow outlets and/or a plurality of overflow outlets. In examples where there are a plurality of fluid inlets and/or a plurality of underflow outlets and/or a plurality of overflow outlets, at least one of the fluid inlet/outlets may be larger than the other, for example having an aperture with a larger cross-sectional area. For example, the separation vessel may comprise a single overflow outlet (for example for waste), and two concentric underflow outlets (for example for taking different cell mass fractions). For example, larger cells/clusters may be extracted through a wider underflow outlet, and smaller/single cells through a narrower underflow outlet.

It will be understood that the inlet fluid control means, the underflow outlet fluid control means and/or the overflow outlet fluid control means may be configured to adjustably vary the flow rate and/or pressure of fluid flowing through the corresponding inlet or outlet, for example based on a predefined relationship between the flow rate through the fluid inlet, the underflow outlet and/or the overflow outlet. For example, the fluid control means may be operable to proportionately increase or decrease the flow rate of fluid through one of the overflow outlet and the underflow outlet based on a proportionate change in flow rate through the fluid inlet—in short, the fluid control means may be operable to variably control the fluid flow rate and/or pressure. The fluid control means may be controller by a controller.

In some examples the system further comprises at least one sensor coupled to a controller and arranged to sense a parameter of the fluid flowing through at least one of the fluid inlet, the underflow outlet and the overflow outlet. In some examples there may also be a sensor inside one of the vessels, such as the separation vessel, the feed vessel and/or the waste vessel. The controller may be configured to make a determination of a parameter of the fluid based on sensor signals received from the at least one sensor, and control at least one of the fluid control means based on the determination/sensor signals received from the at least one sensor.

In some examples the controller may comprise a closed loop control system. For example, the controller may comprise a feedback control loop to control at least one of the fluid control means based on sensor signals received from a sensor as a continuous process. For example, the controller may control at least one of the fluid control means based on sensor signals received from a sensor to maintain the fluid at a point in the system at a particular state (for example to maintain a particular density, impedance, conductance and/or pressure). However, it will be understood that in other examples the controller may be part of an open loop control system.

For example, the system may comprise a first sensor coupled to the controller and arranged to sense a parameter of the fluid flowing through the fluid inlet; and a second (or more) sensor coupled to the controller and arranged to sense a parameter of the fluid flowing through at least one of: (i) the underflow outlet, (ii) the overflow outlet and (iii) inside the separation vessel. In such examples the controller may be configured to control the inlet fluid control means and at least one of: (iv) the underflow outlet fluid control means, and (v) the overflow outlet control means, based on sensor signals received from the sensors.

For example, the controller may be configured to keep the flow rate of fluid through at least one of the outlets above a selected threshold, and if the controller determines that the flow rate falls below the selected threshold, the controller may be operable to control a flub control means to increase the flow rate. Similarly, the controller may additionally or alternatively be configured to control the separation process based on the density and/or size of particles flowing through the fluid through at least one of the fluid inlet, the overflow outlet and/or the underflow outlet. For example, if the controller determines that the density and/or size reaches and/or exceeds a selected threshold, the controller may be operable to control a fluid control means to increase the flow rate through at least one of the underflow and overflow outlets.

The sensors may be selected from at least one of: a turbidity sensor, a temperature sensor, a pressure sensor, a flow sensor, a capacitive sensor and an impedance sensor. The flow sensor may comprise a magnetic flow meter (for example that measurement distortions in an induced magnetic field due to flow), a Coriolis flow meter, an ultrasonic flow meter (for example Doppler flow meter or a time of flight flow meter), and/or a mechanical flow meter (for example using a paddle wheel, turbine or variable flow area). In some examples the sensor may comprise a camera or other similar means for capturing images of the fluid flow, and the camera may be coupled to a processing means such as a computer for processing the images to determine properties such as flow rate. In some examples the sensor may be a capacitive sensor. For example, the sensor may be configured to perform capacitive biomass measurement to determine the viable cell biomass in the fluid by way of generating an electric field in the cell suspension. This causes the cells to polarise, with the more cells being present, the greater the degree of polarisation, which in turn affects the observed capacitance measurement.

In some examples, at least one of the sensors is a turbidity sensor, and the controller may be configured to make a determination of the density of the fluid based on sensor signals received from the turbidity sensor, and control at least one of the fluid control means based on the determined density of the fluid.

In some examples the system further comprises a feed vessel, for example for containing a suspension such as a biological suspension, coupled to the fluid inlet. In use the feed vessel may comprise a suspension, particularly a biological suspension, comprising material such as cells, products secreted or otherwise derived from cells, cell media and/or beads. The controller may be configured to control the pressure of the feed vessel for controlling the flow of fluid through the fluid inlet. For example, the controller may be configured to control a compressed gas feed to the feed vessel to control the pressure in the feed vessel. For example, the controller may be configured to keep the pressure in the feed vessel above a selected threshold, and if the controller determines that the pressure in the feed vessel drops below the selected threshold, the controller may be operable to control the compressed gas feed to supply more compressed gas to the feed vessel to increase the pressure in the feed vessel.

It will be understood, however, that the present invention might also find use in the context of biological suspensions that do not comprise cells (such as during downstream processing of viral vector manufacture processes, once cells have been lysed or otherwise removed). In addition to manufacturing processes, uses will be apparent other fields, such as diagnostics, or any other area where a biological suspension (typically, but not necessarily comprising cells) requires separation.

The fluid control means may comprise energy addition means, such as a pump (for example to control the pressure in a feed, waste or harvest vessel, or directly pumping fluid into or out of the separation vessel). The fluid control means may also comprise energy reduction means, such as a fluid resistor, for example an actuated pinch valve. The fluid control means may also comprise passive means, such as flexible tubes which respond to increased pressure by expanding.

For example, at least one of the fluid control means may comprise a continuous pump such as a rotary pump, a non-continuous pump such as a syringe pump, and/or a fluid resistor (which may comprise flexible tubing that can be adjusted to alter the degree of resistance the tubing provides to the flow of fluid therethrough).

It will also be understood that at least one of the fluid control means may comprise any other means, mechanical or otherwise, capable of controlling the flow of fluid. For example, a fluid control means may comprise a means for controlling the pressure in the system (such as in the feed vessel, harvest vessel or waste vessel as described in more detail below) for controlling the flow of fluid in the system. In such examples the controller may be configured to control the flow rate of fluid by controlling a pressure (either positively or negatively) of the fluid being fed into and/or extracted from the separation vessel. This may comprise, for example, controlling the pressure in the feed vessel, the harvest vessel and/or the waste vessel.

In another aspect there is also provided a separating method, for example a method for separating material, such as cells and/or beads such as alginate beads, from a suspension, particularly from a biological suspension, such as a cell suspension. The method comprises feeding a fluid, for example a suspension (e.g. a biological suspension) containing material such as cells, products secreted or otherwise derived from cells, cell media and/or beads, into a separation vessel via a fluid inlet for establishing a cyclone in the vessel, wherein the separation vessel comprises an underflow outlet, a first overflow outlet and a second overflow outlet. Fluid is fed into the separation vessel transverse to (for example, perpendicular to) and eccentric to a longitudinal axis of the separation vessel such that it creates a cyclone effect in the vessel to form a cyclone about the longitudinal axis of the separation vessel. The method comprises drawing fluid through the underflow outlet and the first overflow outlet and controlling the flow of fluid through the second overflow outlet to control the separation of material from the biological suspension. For example, the separation of media from the biological suspension may be controlled by drawing fluid via the second overflow outlet to increase the concentration of cells in the biological suspension.

The separation vessel may further be coupled to a feed vessel, wherein the fluid inlet, the underflow outlet and the first overflow outlet are coupled to the feed vessel. The separation vessel may further be coupled to a fluid flow control means coupled to the fluid inlet for delivering fluid to the separation vessel from the feed vessel, and a fluid flow control means coupled to the second overflow outlet for removing fluid from the separation vessel. The method may further comprise feeding fluid from the feed vessel into the separation vessel via the fluid inlet and drawing fluid from the separation vessel back to the feed vessel via the underflow outlet and the first overflow outlet.

The method may further comprise adjusting the location at which the second overflow outlet removes fluid from the separation vessel.

The separation vessel has a longitudinal axis, and the method may comprise drawing fluid from the separation vessel via the first overflow outlet at a proximal portion of the longitudinal axis, and drawing fluid from the separation vessel via the underflow outlet at a distal portion of the longitudinal axis, and drawing fluid from the separation vessel via the second overflow outlet at a location between the first overflow outlet and the underflow outlet along the longitudinal axis.

The method may comprise adjusting the location at which the second overflow outlet removes fluid from the separation vessel along the longitudinal axis of the separation vessel.

The method may comprise delivering fluid to the separation vessel via the fluid inlet at a proximal portion of the separation vessel.

The method may comprise drawing fluid from the separation vessel via the first overflow outlet at a distance along the longitudinal axis proximate to the point at which the fluid inlet delivers fluid to the separation vessel.

The first overflow outlet may comprise a tube having a lumen therethrough for drawing fluid from the separation vessel, and the second overflow outlet may comprise a tube having a lumen therethrough for drawing fluid from the separation vessel, and wherein at least a portion of the tube of the second overflow outlet may be configured to sit within at least a portion of the lumen of the first overflow outlet tube, and wherein at least a portion of the second overflow outlet tube may be coaxial with at least a portion of the first overflow outlet tube.

The method may comprise sliding the tube of the second overflow outlet within the lumen of the tube of the first overflow outlet to adjust the location at which the second overflow outlet draws fluid from the separation vessel. The method may comprise adjusting the position of the tube of the second overflow outlet relative to the position of the tube of the first overflow outlet to control the separation of material from the biological suspension. For example, adjusting the position of the tube of the second overflow outlet relative to the position of the tube of the first overflow outlet may control the ratio of medial to cells extracted via the second overflow outlet.

At least a portion of the second overflow outlet tube may be coaxial with at least a portion of the first overflow outlet tube about the longitudinal axis of the separation vessel. The tube of the first overflow outlet may extend into the separation vessel proud of an interior surface of the separation vessel, and wherein the tube of the second overflow outlet may extend into the separation vessel proud of the interior surface of the separation vessel to a greater extent than the tube of the first overflow outlet.

It is noted that while the formation of a cyclone (i.e. the dynamic rotating fluid structure) in the separation system may be preferable, this is not always necessary to achieve separation of denser particles/material from a fluid suspension.

### DRAWINGS

Embodiments of the disclosure will now be described, by way of example only, with reference to the accompanying drawings, in which:

FIG. 1 shows a functional schematic view of an example separating system;

FIG. 2 shows a cross-section through a portion of an example separating system, such as the separating system shown in FIG. 1;

FIG. 3A shows a render of an example separating system such as that shown in FIG. 1;

FIG. 3B shows a photograph of a trial of the example separating system of FIG. 3A using polystyrene beads;

FIG. 4 shows a graph showing the performance of an example separating system, such as the separating system shown in FIGS. 1 and 3A;

FIG. 5 shows a schematic of a separation vessel showing differing parameters of the vessel that may be relevant to cyclone formation;

FIG. 6 shows another functional schematic view of another example separating system; and

FIG. 7 shows another functional schematic view of another example separating system.

### SPECIFIC DESCRIPTION

Embodiments of the disclosure relate to a biological suspension separating system, for example for separating material such as cells from a suspension such as a cell suspension, although it will be understood that the separating system may find application in other fields of use. For example, the separating system might also find use in the context of biological suspensions that do not comprise cells (such as during downstream processing of viral vector manufacture processes, once cells have been lysed or otherwise removed). In addition to manufacturing processes, uses will be apparent other fields, such as diagnostics, or any other area where a biological suspension (typically, but not necessarily comprising cells) requires separation.

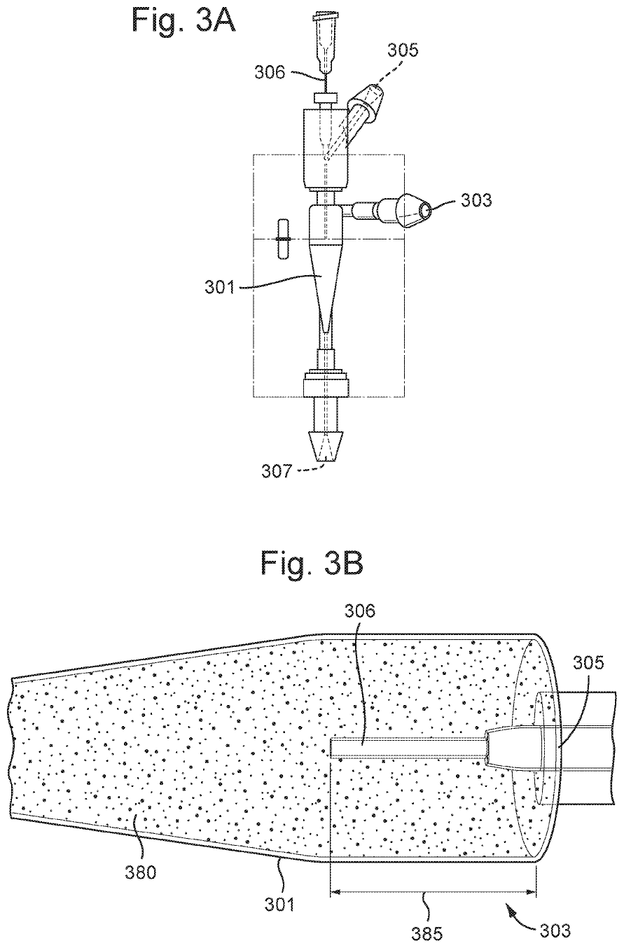

The system involves the use of two overflow outlets and an underflow outlet, wherein the underflow outlet and one of the overflow outlets are coupled to the same feed vessel. Surprisingly, the inventors have discovered that when a separating system is used for the separation of biological suspensions, two vortices are formed inside the separation vessel, one nested within the other and rotating in an opposite direction to the other. What this means is that when the cyclone is formed (comprising both vortices), any material that is suspended in the suspension is forced both upwards along the longitudinal axis of the cyclone towards one of the overflow outlets (by one of the vortices) and downwards along the longitudinal axis of the cyclone towards the underflow outlet (by the other of the vortices). Therefore, to increase the concentration of material such as cells in the suspension, a second overflow outlet is used to extract media f rom the “eye” of the cyclone, as can be seen for example in FIG. 3B.

It will be apparent that the present invention may find use in a great many areas of biotechnology-based industry. One example is for manufacturing applications where populations of cells are grown and used as a means to manufacture other products. In some cases, the cells themselves may in fact be the product of interest. In addition to more long standing cell based manufacturing systems using cells to produce products such as hormones (e.g. insulin or other biologically active factors), secreted proteins, antibodies etc., more recent developments include the production of viruses or viral vector particles for use in the production of vaccines or gene therapies. In the cell and/or gene therapy industries, where yield is extremely important, the use of an optimally tuned separation device is critical and thus the presently developed hydrocyclone will be important to the industry. However, it is noted that the device could also have utility in other industries, particularly those where an increased yield would be desirable.

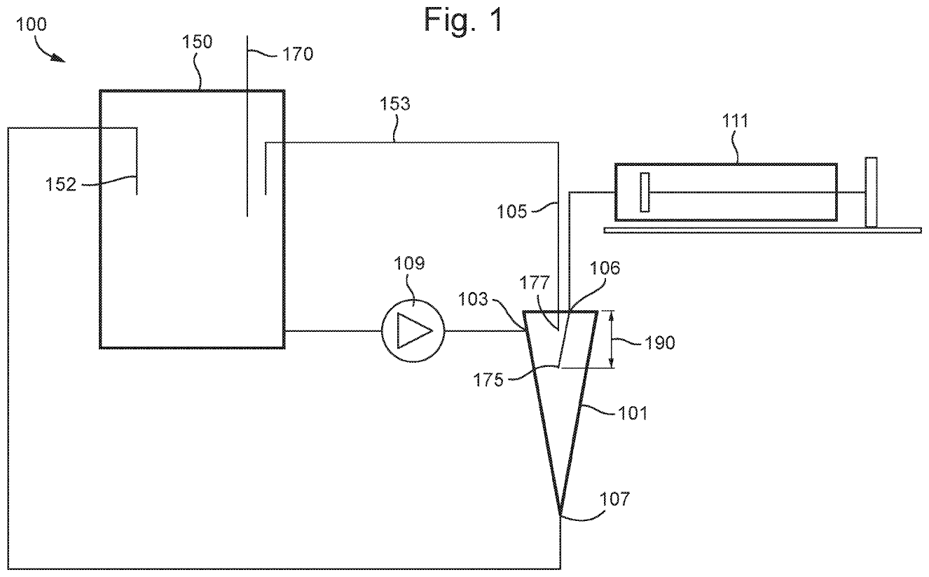

FIG. 1 shows an example separating system 100 of embodiments of the disclosure. The system 100 comprises a separation vessel 101 having a fluid inlet 103, an underflow outlet 107, a first overflow outlet 105 and a second overflow outlet 106. In the example shown the separation vessel 101 is conical in shape to enable the formation of a cyclone therewithin, however it will be understood that in other examples the separation vessel 101 may have another shape. The fluid inlet 103 is proximate to the first overflow outlet 105 and configured to direct fluid into the vessel transverse to (for example, perpendicular to) and eccentric to a longitudinal axis of the conical separation vessel 101 such that it creates a cyclone effect in the vessel about the longitudinal axis. The fluid inlet 103 may be configured to direct fluid into the separation vessel 101 at a position along the longitudinal axis of the vessel 101 at a position below the top of an interior surface of the separation vessel 101, such that a space exists between the fluid inlet 103 and the top of the interior surface of the separation vessel 101 in a direction parallel to the longitudinal axis of the separation vessel 101. The fluid inlet 103 may have a diameter between 1.0 and 4.0 mm. The underflow outlet 107 may have a diameter between 0.1 and 3.0 mm, preferably 0.1 to 1.0 mm, and the first overflow outlet 105 may have a diameter in the range of 0.1 to 3.0 mm, or preferably 0.1 to 1.0 mm.

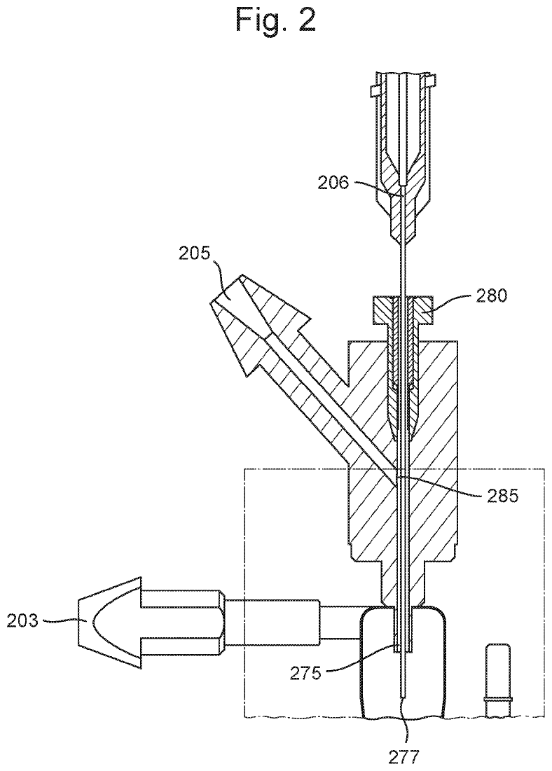

In the example shown the first overflow outlet 105 and the second overflow outlet 106 are at different locations on a top surface of the separation vessel 101. However, in other examples (such as the example shown in FIG. 2) the first overflow outlet 105 and second overflow outlet 106 are coaxial with the longitudinal axis of the conical separation vessel 101. In the example shown in FIG. 1, both the first overflow outlet 105 and the second overflow outlet 106 have respective tubes 177, 175 that extend someway into the separation vessel 101 such that each outlet 105, 106 has a tube 177, 175 that extends proud of the interior surface of the separation vessel 101, wherein each tube has a lumen therethrough for drawing fluid from the separation vessel 101. However, it will be understood that in some examples only the second overflow outlet 106 may have a tube 175 that extends into the separation vessel 101. In the example shown, the tube 175 for the second overflow outlet 106 extends into the separation vessel 101 at an angle relative to the tube 177 for the first overflow outlet 105 and to a greater extent than the tube 177 for the first overflow outlet 105, and extends into the separation vessel 101 by a distance 190. The angle and/or distance 190 at which the tube 175 extends into the separation vessel 101 can be selected, for example, based on a parameter of the fluid flowing through the separation vessel 101 and/or the separation vessel 101 itself (e.g. its geometry such as the underflow diameter (Du), overflow diameter (Do), overflow collar length (Oc), length of cylindrical portion (Lc), diameter of cylindrical portion, and input pressure (p), cone angle, inlet shape, inlet diameter, inlet flow rate, and cyclone scale), such that the location at which fluid is drawn from the separation vessel 101 can be selected to be in an “eye” of a hydrocyclone vortex formed in the vessel 101. In the example shown, the angle and extent to which the tube 175 for the second overflow outlet 106 extends is selected such that the tube 175 draws fluid from the separation vessel a distance 190 from a top interior surface of the separation vessel 101 and a point along the longitudinal axis of the separation vessel, such that both the tube 177 for the first overflow outlet 105 and the tube 175 for the second overflow outlet 175 draw fluid from different points along the longitudinal axis of the separation vessel 101 (the longitudinal axis here also being a line of symmetry of the separation vessel 101).

In some examples the extent to which the tube 175 for the second overflow outlet 106 extends into the separation vessel 101 is adjustable to control the degree and/or mix of fluid drawn from the vessel 101, for example as a function of a parameter of the fluid flowing through the separation vessel 101 (e.g. such as fluid flow rate, volume of separation vessel 101, viscosity of fluid, distance from fluid inlet 103 etc.). For example, the first overflow outlet 105 may be configured to draw fluid, via tube 177, from the separation vessel 101 at a longitudinal extent close to or in line with that of the fluid inlet 103, whereas the second overflow outlet 106 may be configured to draw fluid, via tube 175, from the separation vessel at a longitudinal extent further into the separation vessel 101 and below that of the fluid inlet 103, closer to that of the underflow outlet 107.

In the example shown in FIG. 1, the fluid inlet 103 is coupled to a feed vessel 150 via an inlet fluid control means 109. In the example shown the inlet fluid control means 109 is a continuous pump such as a rotary or peristaltic pump. A centrifugal pump may also be used. A pump with a magnetically suspended impeller, for example, a Levitronix® pump, may be used as it has a low shear and low particle shedding, as well as being pulsation-free. If a peristaltic pump (or any other pulsatile pump) is used it may be coupled to a means to minimise pulsing, for example a flexible tube coupled to the peristaltic pump that is configured to elastically absorb the pulses in pressure in a manner similar to the “windkessel” effect). The first overflow outlet 105 is coupled to the feed vessel 150 via feed lines 153. The underflow outlet 107 is also coupled to the feed vessel 150 via feed line 152. In the example shown the feed vessel 150 also comprises an input line 170. The second overflow outlet 106 is coupled to an outlet fluid control means 111, which in this example is a syringe pump.

In some examples the system 100 also comprises a controller (not shown, but an example of which is described below with reference to FIG. 5) for controlling the system 100, and in particular for controlling the inlet fluid control means 109 and/or the second overflow outlet fluid control means 111.

In the example shown in FIG. 1, which may be used for example for perfusion of biological cells in cell suspensions, the second overflow outlet fluid control means 111 can be used for removing fluid (such as cell media) or particles of lower mass, separated by a cyclone formed in the separation vessel 101. Any higher mass particles (such as cells) would separate out via the underflow outlet 107 and be recycled back into the feed vessel 150, whereas the fluid (such as the cell media) would separate out via the second overflow outlet 106. The input line 170 may be used to replenish any fluid (such as cell media) removed to the waste vessel 125.

The inlet fluid control means 109 is operable to control the flow of fluid though the fluid inlet 103. The second overflow outlet fluid control means 111 is operable to control the flow of fluid through the second overflow outlet 106.

Controlling the flow of fluid through the fluid inlet 103, the underflow outlet 107, the second overflow outlet 106 and/or optionally the first overflow outlet 105 may thus control the formation and functioning of a cyclone in the separation vessel 101.

In use, a fluid (for example a suspension containing cells) is fed into the separation vessel 101 from the feed vessel 150 via the fluid inlet 103 transverse to and eccentric to a longitudinal axis of the separation vessel 101 such that it creates a cyclone effect in the vessel 101. The inlet fluid control means 109 is operated to control the flow rate and/or pressure of fluid fed into the separation vessel 101. The flow of fluid (such as the flow rate and/or pressure) through the second overflow outlet 106 may also be controlled by controlling the second overflow outlet fluid control means 111. Additionally, or alternatively the flow of fluid through the first overflow outlet 105 may be controlled via an optional fluid control means. Controlling the inlet fluid control means 109 and/or the second overflow outlet fluid control means 111 can therefore control the formation of a cyclone and/or a cyclone in the separation vessel 101.

Preferably the flow of fluid through the fluid inlet 103, the first overflow outlet 105 and/or the second overflow outlet 106 is controlled by controlling the inlet fluid control means 109 and/or the respective overflow outlet flow control means, such as the second overflow outlet flow control means 111, such that the flow rate of fluid through the underflow outlet 107 is greater than the flow rate of fluid through the first overflow outlet 105 and/or the second overflow outlet 106.

Once a cyclone is established in the separation vessel 101, in the example of the system 100 being used for cell perfusion, cells may separate out from the separation vessel 101 via the underflow outlet 107 and be fed back (i.e. recycled) into feed vessel 150 via input line 152. Media may separate out from the separation vessel 101 and be extracted by the second overflow outlet fluid control means 111 via the second overflow outlet 106. Cells and media may also separate out from the separation vessel 101 via the first overflow outlet 106 and fed back (i.e. recycled) into feed vessel 150 via input line 153.

The degree to which cells and/or fluid may be separated out may be determined based on at least one of (i) a parameter of the fluid, (ii) time and/or (iii) the position of the second overflow outlet 106 relative to the formed cyclone/vessel 101. For example, control of the second overflow outlet fluid control means 111 may be based on a parameter of the fluid entering and/or in and/or leaving the separation vessel 101 and/or a parameter of the separation vessel 101, such as a parameter or parameters relating to its geometry. Similarly, control of the inlet fluid control means 109 may be based on a parameter of at least one of (i) the fluid entering (ii) the fluid in (iii) the fluid leaving the separation vessel 101 and (iv) a parameter of the separation vessel 101.

For example, if the fluid reaches a selected threshold density (for example, as determined by turbidity), it may be determined that a selected degree of cells should be extracted via the second overflow outlet 106. Additionally or alternatively, the extraction of cells via the second overflow outlet 106 may be a continuous process, and the flow rate of cells extracted via the second overflow outlet 106 may be based on a parameter, such as the density, of fluid entering and/or in and/or leaving the separation vessel 101. In other examples, the extraction of cells via the second overflow outlet 106 may be based on at least one of: (i) levels of toxic by-products (such as lactate or ammonia) from cell metabolism reaching a selected threshold; (ii) cell phenotype changes (for example during differentiation of pluripotent cells); (iii) the size and/or mass of particles such as cells that are desired to be separated from the suspension, for examples particles with a size and/or mass above or below a selected threshold.

The parameter of the fluid may be determined based on fluid entering the fluid inlet 103, fluid passing through the underflow outlet 107 and/or fluid passing through the first overflow outlet 105 and/or the second overflow outlet 106.

Additionally, or alternatively, if a threshold time interval has passed it may be determined to extract a selected amount of media via the second overflow outlet 106, for example where the volume of fluid extracted is determined based on a function of the time interval.

In the example shown in FIG. 1 the pressure of the fluid passing through the fluid inlet 103 may be maintained between 0.5 and 4 bar.

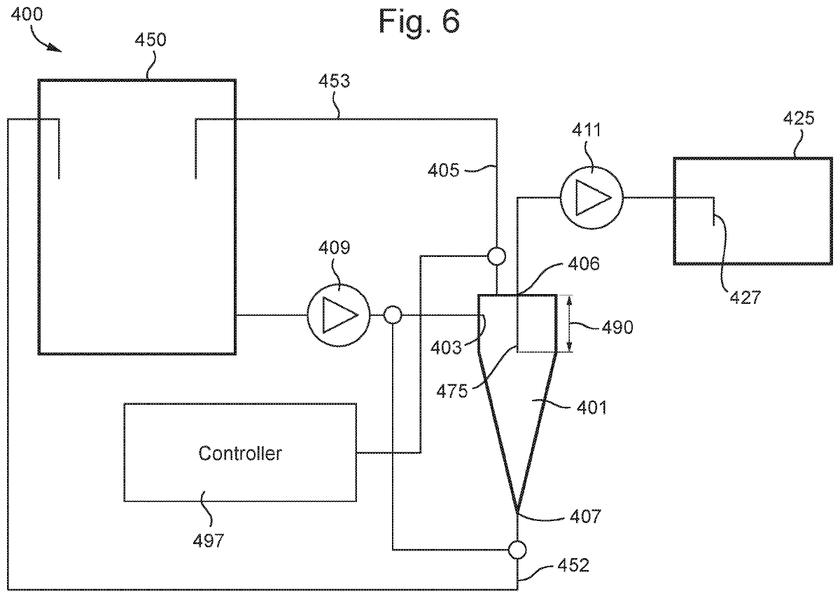

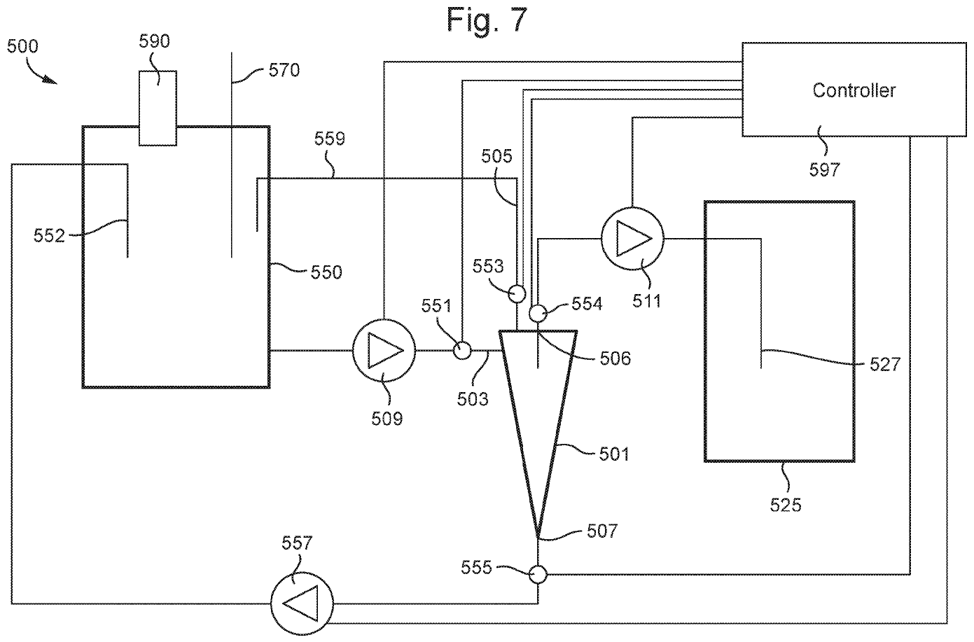

It will be understood that although a controller is not shown in FIG. 1, the functionality described above may be performed by a controller operable to control the inlet fluid control means 109 and the overflow outlet fluid control means 111, as described below with reference to, and as shown in, FIGS. 6 and 7. It will also be understood that the system 100 may comprise sensors coupled to, for example, the fluid inlet 103, the first overflow outlet 105, the second overflow outlet 106 and/or the underflow outlet 107, for sensing the parameter of the fluid discussed above, also as described below with reference to, and as shown in, FIGS. 6 and 7. In some examples there may also be a sensor inside the separation vessel 101 and/or in the feed vessel 150.

It will also be understood that in some examples the system 100 may also comprise an optional underflow outlet fluid control means, also as described below with reference to, and as shown in, FIG. 7.

FIG. 2 shows a cross-section through a portion of an example separating system, such as the separating system shown in FIG. 1. FIG. 2 shows a top portion of the separating system suitable for use, for example, the system shown in FIG. 1. The top portion comprises a fluid inlet 203, a first overflow outlet 205 and a second overflow outlet 206. In the example shown in FIG. 2 the first overflow outlet 205 has a corresponding tube 277 that extends into the separation vessel, and the second overflow outlet 206 has a corresponding tube 275 that also extends into the separation vessel. At least a portion of the tube 277 of the first overflow outlet 205 is coaxial with at least a portion of the tube 275 of the second overflow outlet 206. In the example shown the first overflow outlet 205 and the second overflow outlet 206 are provided as part of a fitting that fits into an aperture in the top of the separation vessel, proximate to the fluid inlet 203. In the example shown, the fitting comprises a 3-way port, wherein the 3-way port has two openings or apertures at a first end (a first and second opening) and a single opening or aperture (a third opening) at a second end opposite to the first end.

In the example shown, the single opening (the third opening) is inserted into an aperture in the separation vessel. The three openings are coupled via respective lumens that join at a junction 285. One of the openings at the first end (the second opening) and the opening at the second end (the third opening) are in line with each along the longitudinal axis of the separation vessel, such that a line can be drawn from the opening at the first end (the second opening) to the opening at the second end (the third opening) along the longitudinal axis. In the example shown, a needle (in this case a 19G needle) is inserted through the opening at the first end (the second opening), along the line drawn to the opening at the second end (the third opening) and into the vessel, wherein the needle provides the tube 275 of the second overflow outlet 206. The other of the openings at the first end (the first opening) is at an angle relative to the longitudinal axis from the junction 285. The internal lumens of the 3-way port have a larger internal diameter than the external diameter of the needle providing the tube 275 of the second overflow outlet 206, such that fluid can flow in the region between the needle/tube 275 of the second overflow outlet 206 and the internal lumens of the 3-way port. A Valco® fitting or plug 280 is inserted into the second opening, and the needle providing the tube 275 of the second overflow outlet 206 is inserted through this Valco® fitting or plug 280 to provide a fluid seal to prevent any fluid flowing between the needle providing the tube 275 of the second overflow outlet 206 and the internal lumens of the 3-way port from flowing out of the second opening. This means that fluid instead flows through the first opening, thus providing the first overflow outlet 205. In this way, the internal lumens of the 3-way port, and specifically the lumen between the first opening at the first end and the third opening at the second end, provide the tube 277 for the first overflow outlet 205. It can be seen from FIG. 2 that the extent to which the 3-way port extends into the aperture of the separation vessel means that the tube 277 for the first overflow outlet 205 extends into the separation vessel along the longitudinal axis of the separation vessel so that it is approximately level with the fluid inlet 203 along the longitudinal axis, but that the needle that provides the tube 275 for the second overflow outlet 206 extends further into the separation vessel, such that it is further into the separation vessel than the tube 277 for the first overflow outlet 205 and the fluid inlet 203.

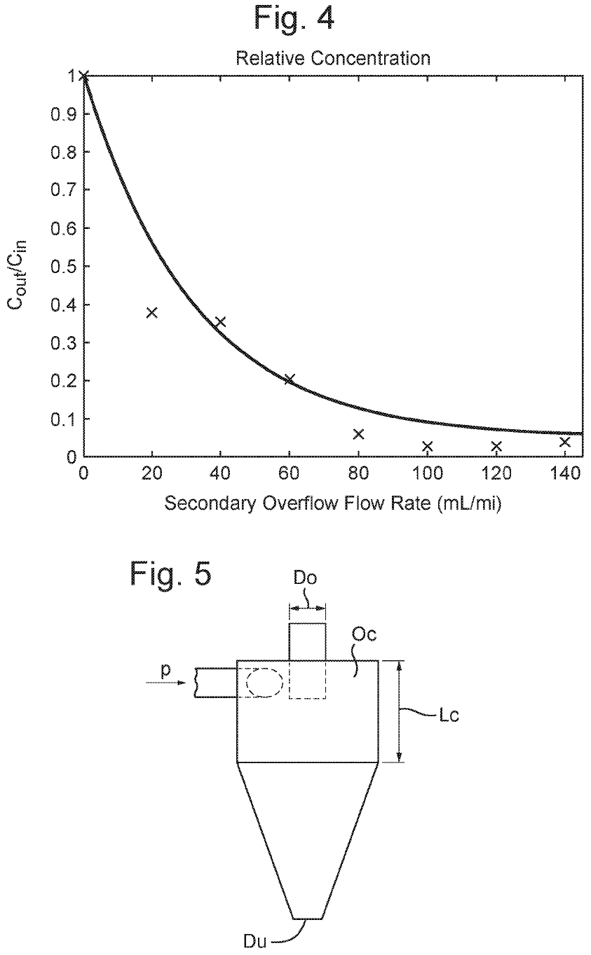

FIG. 3A shows a perspective view of a portion of a separating system comprising the portion of the example separating system shown in FIG. 2. FIG. 3B shows a photograph from a laser imaging study performed using polystyrene beads with the system of FIG. 3A, and FIG. 4 shows a graph showing the results of the laser imaging study. The purpose of the study was to further understand and characterise fluid dynamics of various cyclone configurations using laser imaging. Parameters that were investigated that may affect cyclone formation and/or extraction from the cyclone include underflow diameter (Du), overflow diameter (Do), overflow collar length (Oc), length of cylindrical portion (Lc) and input pressure (p), as shown in FIG. 5. Other parameters that may be relevant include cone angle, inlet shape, inlet diameter, inlet flow rate, cyclone scale and cylindrical diameter.

In the example shown in FIGS. 3A and 3B it can be seen that the separation vessel 303 has a cylindrical portion 385, before it has a conical portion 380 that tapers to the underflow outlet 307. The length of the tube of the second overflow outlet 306 corresponds to that of the length of the cylindrical portion 385 (Lc as indicated in FIG. 5) along the longitudinal axis. The fluid inlet 303 feeds fluid into the separation vessel 301 at the cylindrical portion 385 of the separation vessel 301, and the first overflow outlet 305 draws fluid from the separation vessel 301 at the cylindrical portion 385 of the separation vessel 301 at a point on the longitudinal axis of the separation vessel 301 proximate to the fluid inlet 303.

Polystyrene beads of 5-20 μm in diameter were flushed through a separation vessel at a known input pressure of 1 bar (equivalent to 1 L/min). The separation vessel had the following dimensions: Lc=10 mm; Do=1.5 mm; Du=1.5 mm; Oc=5 mm. The diameter of the cylindrical portion is 10 mm and the cone angle of the conical portion is 8.5 degrees from the central or longitudinal axis of the vessel. The cyclone formed a primary vortex and secondary vortex inside the primary vortex and moving in an opposite direction to the primary vortex (both in direction of travel along the longitudinal axis and rotation about the longitudinal axis). This formed a clarification zone in the middle. The secondary vortex pushed polystyrene beads up through the separation vessel and out through the first overflow outlet 305. A needle tip (providing the tube of the second overflow outlet 306) was placed in the clarification zone providing the second overflow outlet 306. A syringe pump coupled to the second overflow outlet 306 was configured to extract the contents of the clarification zone. Fluid comprising polystyrene beads was fed into the fluid inlet 303 at an input pressure of 1 bar which is equivalent to 1 L/min. Once the laser was activated, the camera captured particle distribution inside the cyclone in the form of a 2D slice image, as shown in FIG. 3B. From this we are able to determine particle distribution/separation. The results of the study are also shown in the graph of FIG. 4.

The secondary overflow outlet 306 is essentially a needle that protrudes to the inner core of the cyclone at one end and connected to a fluid control means, such as a syringe pump (as shown in FIG. 1) at the other. If there is separation of particles within the cyclone then the contents of the syringe pump would mostly be waste i.e. media. We can analyse this by measuring, for example, the particle or cell concentration going into the cyclone (Cᵢₙ) and particle or cell concentration in the secondary overflow/syringe pump (Cₒᵤₜ). The results of this are shown in FIG. 4. The graph of FIG. 4 shows that as the flow rate on the syringe pump is increased, a much cleaner waste is extracted from the cyclone. At 80 mL/min we are seeing around 95% separation of polystyrene beads.

This experimental setup was also repeated using Jurkat cells. RPMI 1640 cell culture medium was used supplemented with 10% foetal bovine serum. Cells were grown in T75 flasks until they reached a concentration of ˜1e6/mL. Cells were then passaged into T175 flasks and grown to reach the same desired cells were then taken from the flasks and placed inside a 1000 mL bag which was used as the feed vessel. The cell culture bag has a sample port which we used to extract 1 mL of cell containing media. The bag was tilted before use slightly left and right to make sure a homogenous spread of cells was achieved. Material from the secondary overflow was collected in a 60 mL syringe. The syringe was slowly swirled to make sure cells and other components of the media are evenly spread. A cell viability assay was conducted using the nucleocounter. This assessed the total cell concentration, viable and also dead cell concentration. However, it is primarily the viable cell concentration that is of interest. The study showed that as the flow rate on the syringe pump was increased, a much cleaner waste was extracted from the cyclone.

It is envisioned that a separation vessel used for separation biological suspensions is much smaller than a separation vessel used in other industries, for example, in the oil and gas field. Accordingly, a biological suspension separation vessel will likely have maximum dimensions, such as: P=2 bar, equivalent to 2 L/min (although higher shear stresses may have an adverse effect on cell viability); Lc=10 mm; Do=2.5 mm; Du=3.5 mm; Oc=10 mm; diameter of cylindrical portion=10 mm; cone angle=8.5 degrees.

FIG. 6 shows another example separating system 400 of embodiments of the disclosure and is similar to the system described above with reference to FIG. 1 with like reference numbers indicating similar or the same entities. However, the system of FIG. 6 is slightly different in that it has a cylindrical portion at a proximate portion of the separation vessel 401, before the separation vessel 101 has a conical portion that tapers to the underflow outlet 407, similar to the separation vessel shown in FIGS. 3A and 3B. In the example shown the fluid inlet 403 is provided in the cylindrical portion of the separation vessel 401.

As with the example shown in FIG. 1, in the example of FIG. 6 both the first overflow outlet 405 and the second overflow outlet 406 have respective tubes, wherein each tube has a lumen therethrough for drawing fluid from the separation vessel 401. In the example shown, the tube 475 for the second overflow outlet 406 extends into the separation vessel 401 by a distance 490. In the example shown, the distance 490 at which the tube 475 extends into the separation vessel 401 is selected based on a parameter of the separation vessel 401 itself—in this case the length (in the longitudinal direction) of the cylindrical portion of the separation vessel 401, such that the location at which fluid is drawn from the separation vessel 401 can be selected to be in an “eye” of a hydrocyclone vortex formed in the vessel 401.

In the example shown in FIG. 6, the fluid inlet 403 is coupled to a feed vessel 450 via an inlet fluid control means 409. In the example shown the inlet fluid control means 409 is a continuous pump such as a peristaltic or centrifugal pump. The second overflow outlet 406 is coupled to a waste vessel 425 via an overflow outlet fluid control means 411 and a waste line 427. The second overflow outlet fluid control means 411 is also a continuous pump such as a peristaltic pump. The underflow outlet 407 is coupled to the feed vessel 450 via a feed line 452, and the first overflow outlet 405 is also coupled to the harvest vessel 450 via a feed line 453

In the example shown the system 400 also comprises a controller 497 for controlling the system 400, and in particular for controlling the inlet fluid control means 409 and the overflow outlet fluid control means 411.

In the example shown in FIG. 6, which as noted above, may be used for example for cell harvest, the waste vessel 425 can be used for removing less dense fluid (such as cell media) separated by a cyclone formed in the separation vessel 401. Any higher density particles (such as cells) would separate out via the underflow outlet 407 into harvest vessel 408.

The inlet fluid control means 409 is operable to control the flow of fluid though the fluid inlet 403. The second overflow outlet fluid control means 411 is operable to control the flow of fluid through the second overflow outlet 406. Controlling the flow of fluid through the fluid inlet 403 and/or the second overflow outlet 406 may thus control the formation and functioning of a cyclone in the separation vessel 401.

In use a fluid (for example a biological suspension containing cells) is fed into the separation vessel 401 transverse to and eccentric to the longitudinal axis of the separation vessel 401 from the feed vessel 450 via the fluid inlet 403. The inlet fluid control means 409 is controlled to control the flow rate and/or pressure of fluid fed into the separation vessel 401. The flow of fluid (such as the flow rate and/or pressure) through the second overflow outlet 406 is also controlled by controlling the second overflow outlet fluid control means 411. Controlling the inlet fluid control means 409 and/or the second overflow outlet fluid control means 411 can therefore control the formation of the cyclone in the separation vessel 401.

In some examples the flow rate of the second overflow outlet fluid control means 411 is controlled to achieve a desired degree of separation of cells. For example, the second overflow outlet fluid control means may be controlled to operate at a minimum threshold flow rate so that predominantly media and not cells are extracted via the second overflow outlet 406.

Preferably the flow of fluid through the fluid inlet 403 and the second overflow outlet 405 is controlled by controlling the inlet fluid control means 409 and/or the overflow outlet flow control means 411 such that the flow rate of fluid through the underflow outlet 407 is greater than the flow rate of fluid through the second overflow outlet 406.

Once a cyclone is established in the separation vessel 401, in the example of the system 400 being used for cell harvest, cells may separate out from the separation vessel 401 via both the underflow outlet 407 (via a first vortex) and first overflow outlet 405 (via a second vortex), and into harvest vessel 408. Cell media may separate out from the separation vessel 401 and be extracted by the overflow outlet fluid control means 411 via the second overflow outlet 406 and into waste vessel 425.

The degree to which fluid is separated out into the waste vessel 425 may be determined based on a parameter of the fluid and/or time. For example, control of the overflow outlet fluid control means 411 may be based on a parameter of the fluid entering and/or in and/or leaving the separation vessel 401. Similarly, control of the inlet fluid control means 409 may be based on a parameter of the fluid entering and/or in and/or leaving the separation vessel 401. The parameter of the fluid may be determined based on fluid entering the fluid inlet 403, fluid passing through the underflow outlet 407 and/or fluid passing through the second overflow outlet 406.

FIG. 7 shows another example separating system 500 of embodiments of the disclosure and is similar to the system described above with reference to FIGS. 1 to 6 with like reference numbers indicating similar or the same entities. However, the system of FIG. 7 also has an optional controller 597 for controlling operation of the system 500 and optional sensors 551, 553, 554 and 555.

As with the system of FIG. 1, the system 500 shown in FIG. 7 comprises a separation vessel 501 having a fluid inlet 503, an underflow outlet 507, a first overflow outlet 505 and a second overflow outlet 506. The separation vessel 501 is conical in shape to enable the formation of a cyclone therewithin, but it will be understood that as with the example shown in FIG. 6 it may further comprise a cylindrical portion at a proximal end of the separation vessel. The fluid inlet 503 is proximate to the first overflow outlet 505 and configured to direct fluid into the separation vessel 501 transverse to and eccentric to the longitudinal axis of the conical vessel 501. The underflow outlet 507 and optionally at least one of the overflow outlets 505, 506 may be coaxial with the longitudinal axis of the conical separation vessel 501.

In the example shown in FIG. 7, the fluid inlet 503 is coupled to a feed vessel 550 via an inlet fluid control means 509. In the example shown the inlet fluid control means 509 is a continuous pump such as a peristaltic or centrifugal pump. The first overflow outlet 505 is coupled to the feed vessel 550 via a feed line 559, and the second overflow outlet 506 is coupled to a waste vessel 525 via an overflow outlet fluid control means 511 and a waste line 527. The overflow outlet fluid control means 511 is also a continuous pump such as a peristaltic pump. The underflow outlet 507 is also coupled to the feed vessel 550 via an underflow outlet fluid control means 557 and a feed line 552. In the example shown the feed vessel 550 also comprises an input line 570 and a valve 590. Although not shown in FIG. 7, in some examples the first overflow outlet 505 may also be coupled to an overflow outlet fluid control means for controlling the flow of fluid to the feed vessel 550 via the feed line 559 and the first overflow outlet 505.

Sensors are also coupled to the input and outputs of the separation vessel 501. An inlet sensor 551 is coupled to the fluid inlet 503, a first overflow sensor 553 is coupled to the first overflow outlet 505, a second overflow sensor 554 is coupled to the second overflow outlet 506 and an underflow sensor 555 is coupled to the underflow outlet 507. The sensors 551, 553, 554, 555 may be selected from at least one of: a turbidity sensor, a temperature sensor, a pressure sensor, a capacitive sensor and an impedance sensor.

The system 500 also comprises a controller 597 for controlling the system 500. The controller 597 is coupled to the inlet fluid control means 509, the overflow outlet fluid control means 511 and the underflow outlet fluid control means 557. The controller 597 is also coupled to the inlet sensor 551, the first overflow sensor 553, the second overflow sensor 554 and the underflow sensor 555. The controller is also coupled to valve 590.

In the example shown in FIG. 7, which may be used for example for perfusion of biological cells in cell suspensions, the waste vessel 525 can be used for removing less dense fluid (such as cell media) separated by a cyclone formed in the separation vessel 501. Any higher density particles (such as cells) would separate out via the underflow outlet 507 and the first overflow outlet 505 and be recycled back into the feed vessel 550, whereas the less dense fluid (such as the cell media) would separate out via the second overflow outlet 506 and into the waste vessel 525. The input line 570 may be used to replenish any fluid (such as cell media) removed to the waste vessel 525.

The inlet fluid control means 509 is operable to control the flow of fluid though the fluid inlet 103. The overflow outlet fluid control means 511 is operable to control the flow of fluid through the second overflow outlet 506. The underflow outlet fluid control means 557 is operable to control the flow of fluid through the underflow outlet 557. The valve 590 may be controlled to control the pressure in the feed vessel 550, and thus the pressure of fluid flowing into the separation vessel 501.

The inlet sensor 551 is operable to sense a parameter of the fluid flowing through the fluid inlet 503. The first overflow sensor 553 is operable to sense a parameter of fluid flowing through the first overflow outlet 505, and the second overflow sensor 554 is operable to sense a parameter of the fluid flowing through the second overflow outlet 506. The first overflow sensor 553 is operable to sense a parameter of fluid flowing through the overflow outlet 505. The underflow sensor 555 is operable to sense a parameter of fluid flowing through the underflow outlet 507.

The controller 597 is operable to control the inlet fluid control means 509, the overflow outlet fluid control means 511, the underflow outlet fluid control means 557 and optionally valve 590 to control the flow of fluid into and out of the separation vessel 501. The valve 590 may be operable to control the pressure in the feed vessel 590 and therefore the pressure of fluid flowing into the separation vessel 501, for example by introducing a fluid such as a gas into the feed vessel 590, or allowing a pressurised fluid such as a gas to escape the feed vessel 590. It will be understood that in some examples the harvest vessel and/or waste vessel (if present) may also comprise a similar valve.

The controller 597 is also operable to control to the inlet sensor 551, the first overflow sensor 553, the second overflow sensor 554 and the underflow sensor 555. The inlet sensor 551, the first overflow sensor 553, the second overflow sensor 554 and the underflow sensor 555 are configured to send sensor signals indicative of a parameter of the fluid to the controller 597. The controller 597 is configured to make a determination of a parameter of the fluid based on the received sensor signals.

In use a fluid (for example a biological suspension containing cells) is fed into the separation vessel 501 transverse to and eccentric to the longitudinal axis of the separation vessel 501 from the feed vessel 550 via the fluid inlet 503. The inlet fluid control means 509 is controlled by the controller 597 to control the flow rate and/or pressure of fluid fed into the separation vessel 501. The flow of fluid (such as the flow rate and/or pressure) through the second overflow outlet 506 is also controlled by the controller 597 by controlling the overflow outlet fluid control means 511 and/or the underflow outlet fluid control means 557. Controlling the inlet fluid control means 509 and/or the overflow outlet fluid control means 511 and/or underflow outlet fluid control means 557 can therefore control the formation of the cyclone in the separation vessel 501.

Preferably the flow of fluid through the fluid inlet 503 and the second overflow outlet 506 is controlled by controlling the inlet fluid control means 509 and/or the overflow outlet flow control means 511 such that the flow rate of fluid through the underflow outlet 507 is greater than the flow rate of fluid through the second overflow outlet 506.

Once a cyclone is established in the separation vessel 501, in the example of the system 500 being used for cell perfusion, cells may separate out from the separation vessel 501 via the underflow outlet 507 and the first overflow outlet 505 and be fed back (i.e. recycled) into feed vessel 550 via input line 552. Waste media may separate out from the separation vessel 501 and be extracted by the overflow outlet fluid control means 511 via the second overflow outlet 506 and into waste vessel 525.

In the example shown in FIG. 7, control of the fluid control means, such as the inlet fluid control means 509, the overflow outlet fluid control means 511 and/or the underflow outlet fluid control means 557 is based on a parameter of the fluid entering and/or in and/or leaving the separation vessel 501. Control of the valve 590 may also be based on a parameter of the fluid entering and/or in and/or leaving the separation vessel 501. This is done by the controller 597 controlling operation of the inlet sensor 551, the overflow sensor 553 and the underflow sensor 555 to receive sensor signals indicative of a parameter of the fluid at those points. The controller 597 makes a determination of a parameter of the fluid based on the received sensor signals, and determines what control of the inlet fluid control means 509, the overflow outlet fluid control means 511, the underflow outlet fluid control means 557 and/or valve 590 is needed based on the determined parameters of the fluid.

For example, if the fluid entering the separation vessel 501 reaches a selected threshold density (for example, as determined by inlet sensor 551 which may be a turbidity sensor), it may be determined by the controller that fluid should be extracted via the second overflow outlet 506. The amount of fluid that is extracted may be based on the determined density of the fluid, for example the amount of fluid extracted may be proportional to the difference between the measured density and the threshold density. This may be done by controlling operation of the overflow outlet control means 511 and/or the underflow outlet control means 557.

Additionally or alternatively, the extraction of fluid via the second overflow outlet 506 may be a continuous process, and the flow rate of fluid extracted via the second overflow outlet 506 may be based on a parameter, such as the density, of fluid entering and/or in and/or leaving the separation vessel 501. For example, the controller may have a feedback control loop that continuously monitors a parameter of the fluid (such as the density) and controls operation of the overflow outlet control means 511 as a continuous process based on the feedback control loop.

Additionally, or alternatively, if a threshold time interval has passed it may be determined to extract a selected amount of fluid via the second overflow outlet 506, for example where the volume of fluid extracted is determined based on a function of the time interval. For example, the controller 597 may extract a selected amount of fluid repeatedly at a selected time interval via the second overflow outlet 506 by controlling the overflow outlet fluid control means 511.

It will be understood that although the system 500 shown in FIG. 7 comprises an inlet fluid control means 509, an overflow outlet fluid control means 511 and an underflow outlet fluid control means 557, it will be understood that in some examples the system 500 may not comprise all three control means or may comprise more control means (for example the first overflow outlet 505 may also comprise a fluid control means for controlling the flow of fluid from the separation vessel 501 via the first overflow outlet 505 back to the feed vessel 550. In addition, although the system 500 shown in FIG. 7 comprises an inlet sensor 551, a first overflow sensor 553, a second overflow sensor 554 and an underflow sensor 555, in some examples the system 500 may only comprise two or even only one sensor. In addition, it will be understood that the valve 590 is optional.

The controller 597 may be configured to operate in two modes:
(i) an initialisation mode for establishing a cyclone in the separation vessel; and
(ii) a cyclone mode for separating material from suspensions, e.g. cells from a suspension.

In the initialisation mode the controller 597 may be configured to inhibit the flow of fluid through the first overflow outlet 505 and/or second overflow outlet 506, but only, for example, through the fluid inlet 503 and/or the underflow outlet 507. In some examples, in the initialisation mode the controller 597 may be configured to control the flow of fluid through the first overflow outlet 505 such that no fluid flows through the first overflow outlet 505 (for example, so that it is blocked).

In the cyclone mode the controller 597 may be configured to adjustably control the flow of fluid through at least one of (i) the first overflow outlet 505, (ii) the second overflow outlet 506 and (iii) the underflow outlet 507. The controller 597 may be configured to determine when to switch between the initialisation mode and the cyclone mode based on a parameter, such as the speed and/or pressure, of the fluid passing through at least one of (i) the underflow outlet 507, (ii) the first overflow outlet 505 and (iii) the second overflow outlet 506. Preferably the controller 597 is configured to switch between the initialisation mode and the cyclone mode based on a parameter of the fluid passing through the first overflow outlet 505 and/or second overflow outlet 506. When the controller 597 switches between the initialisation mode and the cyclone mode, the controller 597 may be configured to gradually increase or ramp up the flow rate of the flow of fluid through the first overflow outlet 505 to a selected rate, for example from a flow rate of zero (i.e. blocked) to the selected flow rate. This may be desirable so as not to create any sudden/destabilising perturbations to the system which may result in the cyclone collapsing.

The examples described above and as shown in FIGS. 1 to 7 only have one fluid inlet, however, it will be understood that in other examples the separation vessel may have a plurality of fluid inlets. Each of the plurality of fluid inlets may have a respective fluid inlet control means. Having a plurality of fluid inlets each with a respective fluid inlet control means may allow the formation of a cyclone in the separation vessel to be more easily controlled. For example, the separation vessel may have a first fluid inlet configured to feed fluid in a first direction transverse to, and eccentric to the longitudinal axis of the separation vessel on one side of the separation vessel, and a second fluid inlet configured to feed fluid in a second direction opposite to the first direction and transverse to, and eccentric to the longitudinal axis of the separation vessel on an opposing side of the separation vessel. This may be beneficial as it may reduce the asymmetry of energy input at the top of the cyclone formed in the separation vessel, which may increase the speed at which a cyclone forms in the separation vessel, and the stability of a cyclone formed in the separation vessel.

As noted above, it will also be understood that the separation vessel may comprise a plurality of underflow and/or more than two overflow outlets (optionally with respective fluid control means) and each coupled to a tube or line with a corresponding bore or diameter matching that of the respective outlet. In some examples the plurality of underflow and/or overflow outlets may have differing diameters and may be concentric with each other—for example, if there are two overflow outlets, one with a larger bore or diameter than the other, the two overflow outlets may be concentric with each other (for example such that one sits inside the other).

Although all of the examples shown above with reference to FIGS. 1 to 7 show some form of inlet fluid control means, it will be understood that in some examples an inlet fluid control means is not essential. For example, a fluid suspension may be into the separation vessel under gravity if the feed vessel is positioned above the separation vessel.

It will be appreciated that in the context of the examples described above the fluid is a liquid comprising biological material suspended in a suspension. However, it will be appreciated that embodiments described herein may be used for removing small or powdered solids from air, water, or other gases or liquids by centrifugal force.

It will be appreciated from the discussion above that the embodiments shown in the FIGS. are merely exemplary, and include features which may be generalised, removed or replaced as described herein and as set out in the claims. In the context of the present disclosure other examples and variations of the apparatus and methods described herein will be apparent to a person of skill in the art.

1. A biological suspension separating system for separating material from a biological suspension, the system comprising:
a separation vessel arranged to enable the formation of a cyclone therewithin, the separation vessel comprising:
a fluid inlet for delivering fluid to the vessel;
an underflow outlet for removing fluid from the vessel;
a first overflow outlet configured to remove fluid from a first region inside the separation vessel; and
a second overflow outlet configured to remove fluid from a second region inside the separation vessel.

2. The system of claim 1 further comprising:
a feed vessel, wherein the fluid inlet, the underflow outlet and the first overflow outlet are coupled to the feed vessel; and
a fluid flow control coupled to the fluid inlet and configured to deliver fluid to the separation vessel from the feed vessel; and
a fluid flow control coupled to the second overflow outlet and configured to remove fluid from the separation vessel.

3. The system of claim 1 wherein a location at which the second overflow outlet removes fluid from the vessel is adjustable.

4. The system of claim 1, wherein the separation vessel has a longitudinal axis, and wherein the first overflow outlet is configured to draw fluid from the separation vessel at a proximal portion of the longitudinal axis and the underflow outlet is configured to draw fluid from the separation vessel at a distal portion of the longitudinal axis, and wherein the second overflow outlet is configured to draw fluid from the separation vessel at a location between the first overflow outlet and the underflow outlet along the longitudinal axis.

5. The system of claim 4 wherein the location at which the second overflow outlet removes fluid from the separation vessel is adjustable along the longitudinal axis of the separation vessel.

6. The system of claim 4 wherein the fluid inlet is configured to deliver fluid to the separation vessel at a proximal portion of the separation vessel, wherein the first overflow outlet is configured to draw fluid from the separation vessel at a distance along the longitudinal axis proximate to the point at which the fluid inlet delivers fluid to the separation vessel.

7. (canceled)

8. The system of claim 1 wherein the first overflow outlet comprises a tube having a lumen therethrough for drawing fluid from the separation vessel, and wherein the second overflow outlet comprises a tube having a lumen therethrough for drawing fluid from the separation vessel, and wherein at least a portion of the tube of the second overflow outlet is configured to sit within at least a portion of the lumen of the first overflow outlet tube, and wherein at least a portion of the second overflow outlet tube is coaxial with at least a portion of the first overflow outlet tube.

9. The system of claim 8 wherein the tube of the second overflow outlet is slidable within the lumen of the tube of the first overflow outlet.

10. The system of claim 8 or 9 wherein the position of the tube of the second overflow outlet is adjustable relative to the position of the tube of the first overflow outlet.

11. The system of claim 8, wherein at least a portion of the second overflow outlet tube is coaxial with at least a portion of the first overflow outlet tube about a longitudinal axis of the separation vessel.

12. The system of claim 8 wherein the tube of the first overflow outlet extends into the separation vessel proud of an interior surface of the separation vessel, and wherein the tube of the second overflow outlet extends into the separation vessel proud of the interior surface of the separation vessel to a greater extent than the tube of the first overflow outlet.

13. A method for separating material from a biological suspension, the method comprising:
feeding a biological fluid suspension containing material into a separation vessel via a fluid inlet to establish a cyclone in the separation vessel about a longitudinal axis of the separation vessel;
wherein the vessel comprises:
an underflow outlet;
a first overflow outlet; and
a second overflow outlet;
wherein the fluid is fed into the separation vessel transverse to the longitudinal axis of the separation vessel; and
drawing fluid through the underflow outlet and the first overflow outlet and controlling a flow of fluid through the second overflow outlet to control a separation of material from the biological suspension.

14. The method of claim 13, wherein the separation vessel is coupled to:
a feed vessel, wherein the fluid inlet, the underflow outlet and the first overflow outlet are coupled to the feed vessel;
a fluid flow control means coupled to the fluid inlet for delivering fluid to the separation vessel from the feed vessel; and
a fluid flow control means coupled to the second overflow outlet for removing fluid from the separation vessel;
wherein the method further comprises feeding fluid from the feed vessel into the separation vessel via the fluid inlet and drawing fluid from the separation vessel back to the feed vessel via the underflow outlet and the first overflow outlet.

15. The method of claim 13 further comprising adjusting a location at which the second overflow outlet removes fluid from the separation vessel.

16. The method of claim 13, wherein the separation vessel has a longitudinal axis, the method comprising drawing fluid from the separation vessel via the first overflow outlet at a proximal portion of the longitudinal axis, and drawing fluid from the separation vessel via the underflow outlet at a distal portion of the longitudinal axis, and drawing fluid from the separation vessel via the second overflow outlet at a location between the first overflow outlet and the underflow outlet along the longitudinal axis, further comprising adjusting the location at which the second overflow outlet removes fluid from the separation vessel along the longitudinal axis of the separation vessel.

17. (canceled)

18. The method of claim 16 comprising delivering fluid to the separation vessel via the fluid inlet at a proximal portion of the separation vessel, further comprising drawing fluid from the separation vessel via the first overflow outlet at a distance along the longitudinal axis proximate to the point at which the fluid inlet delivers fluid to the separation vessel.

19. (canceled)

20. The method of claim 13 wherein the first overflow outlet comprises a tube having a lumen therethrough for drawing fluid from the separation vessel, and wherein the second overflow outlet comprises a tube having a lumen therethrough for drawing fluid from the separation vessel, and wherein at least a portion of the tube of the second overflow outlet is configured to sit within at least a portion of the lumen of the first overflow outlet tube, and wherein at least a portion of the second overflow outlet tube is coaxial with at least a portion of the first overflow outlet tube.

21. The method of claim 20 comprising at least one of (i) sliding the tube of the second overflow outlet within the lumen of the tube of the first overflow outlet to adjust a location at which the second overflow outlet draws fluid from the separation vessel, and (ii) adjusting the position of the tube of the second overflow outlet relative to the position of the tube of the first overflow outlet to control the separation of material from the biological suspension.

22. (canceled)

23. The method of claim 20, wherein at least a portion of the second overflow outlet tube is coaxial with at least a portion of the first overflow outlet tube about the longitudinal axis of the separation vessel.

24. The method of claim 20 wherein the tube of the first overflow outlet extends into the separation vessel proud of an interior surface of the separation vessel, and wherein the tube of the second overflow outlet extends into the separation vessel proud of the interior surface of the separation vessel to a greater extent than the tube of the first overflow outlet.
